(19) United States
(12) Patent Application Publication
(10) Pub. No.: US 2008/0312253 A1
(43) Pub. Date: Dec. 18, 2008

(54) Pharmaceutical compositions containing pyrazole derivatives for treating as serotonin antagonist

(75) Inventors: Ghil Soo Nam, Seoul (KR); Hye Whon Rhim, Seoul (KR); Ae Nim Pae, Seoul (KR); Kyung II Choi, Seoul (KR); Hyun Ah Choo, Seoul (KR); Seung Yeol Nah, Seoul (KR)

Correspondence Address: DICKINSON WRIGHT PLLC, 1875 Eye Street, NW, Suite 1200, WASHINGTON, DC, 20006, US

(73) Assignee: KOREA INSTITUTE OF SCIENCE AND TECHNOLOGY, Seoul (KR)

(21) Appl. No.: 12/213,067

(22) Filed: Jun. 13, 2008

(30) Foreign Application Priority Data
Jun. 14, 2007 (KR) 10-2007-0058542

(51) Int. Cl. A61K 31/496; A61P 25/16; A61P 25/28; A61P 25/18

(52) U.S. Cl. 514/253.09; 514/254.07; 514/254.05

(57) ABSTRACT

The present invention relates to a pharmaceutical composition containing a pyrazole derivative as an active ingredient, which has antagonistic activity against serotonin 5-HT₃A and is effective for the prevention and treatment of central nervous system (CNS) diseases, including emesis, nausea, alcoholism, drug abuse, depression, compulsive neurosis, anxiety, seizure, Alzheimer's disease, Parkinson's disease, Huntington's chorea, psychosis, schizophrenia, suicidal tendency, sleep disorder, appetite disorder and migraine.

### CROSS-REFERENCE TO RELATED APPLICATION

This application claims the benefit of Korean Patent Application No. 10-2007-0058542, filed Jun. 14, 2008, pursuant to 35 USC § 119 (a), the entire contents of which are incorporated herein by reference.

### BACKGROUND OF THE INVENTION

1. Field of the Invention

The present invention relates to a pharmaceutical composition containing a pyrazole derivative as an active ingredient, which has antagonistic activity against serotonin 5-HT₃A and is effective for the prevention and treatment of central nervous system (CNS) diseases, including emesis, nausea, alcoholism, drug abuse, depression, compulsive neurosis, anxiety, seizure, Alzheimer's disease, Parkinson's disease, Huntington's chorea, psychosis, schizophrenia, suicidal tendency, sleep disorder, appetite disorder and migraine, for example.

2. Description of the Background

Serotonin is known to play an important role in psychiatric disorders (e.g., depression, aggression, seizure, compulsive neurosis, psychosis, schizophrenia, suicidal tendency), degenerative nerve disorders (e.g., Alzheimer's disease, Parkinson's disease, Huntington's chorea, anorexia, polyphagia, insomnia, alcoholism-related disorders, cerebral vascular accidents, migraine, and various other pathologic conditions [Meltzer, Neuropsychopharmacology, 21:106S-115S (1999); Barnes & Sharp. Neuropsychopharmacology, 38:1083-1152 (1999): Glennon, Neurosci. Biobehavioral Rev., 14:35 (1990)]. Serotonin (5-hydroxytryptamine, or 5-HT) receptors are present in human and animals, and play an important role in physiological and behavioral functions. Until now, about 15 genetically different 5-HT receptor subtypes have been cloned. Each subtype exhibits unique distribution and shows various preference and relationships for different ligands.

A serotonin 5-HT₃ receptor is a ligand-gated ionotropic receptor which allows the passage of cations [Maricq et al, Science, 1991, 254, 432-437]. The 5-HT₃ receptor is mainly found in the human CNS [Morales, M et al, J. Neurosci., 2002, 22, 6732-6741]. The 5-HT₃A receptor subtype was first cloned in 1991 by Maricq et al [Maricq et al, Science, 1991, 254, 432-437], and is found in the peripheral and limbic areas of the brain, including cortex, amygdala, hippocampus, and so forth [Tecott et al, Pro. Natl. Acad. Sci. U.S.A. 1993, 90, 1430-1434].

The 5-HT₃ receptor exists either as 5-HT₃A homomer or 5-HT₃A and 5-HT₃B heteromer. Both have the 5-HT₃A subtype and it is known that their function is mostly provided by 5-HT₃A. According to physiological studies on the 5-HT₃A receptor, ondansetron, granisetron, tropisetron, and the like are effective and selective antagonists of the receptor [Gaster et al, Med. Res. Rev. 1997, 17, 163-214]. Clinical research on these compounds indicates that they are effective in the treatment or amelioration of emesis or nausea, for example, during cancer chemotherapy. Further, the 5-HT₃A receptor is associated with alcoholism, drug abuse, depression, cognitive performance, psychological anxiety, pain, and the like [Silverston et al, Exp. Opi. Ther. Patents 1996, 6, 471-481].

### SUMMARY OF THE INVENTION

In accordance with the present invention, it has been discovered that pyrazole compounds, i.e., pyrazole derivatives, disclosed in the literatures Korean Patent No. 0654328; and U.S. patent application Ser. No. 11/50,769, exhibit pharmacological antagonistic activity against serotonin 5-HT₃A, and that these compounds provide superior preventive and therapeutic effects in related CNS diseases.

Accordingly, an object of the present invention is to provide a pharmaceutical composition that can be used in preventing and treating of CNS diseases involving serotonin 5-HT₃A.

### DETAILED DESCRIPTION OF THE PREFERRED EMBODIMENTS

In one aspect, the present invention provides a pharmaceutical composition for the prevention and treatment of CNS diseases related to the serotonin 5-HT₃A receptor, which contains one or more pyrazole derivatives represented by the following Formula 1 or pharmaceutically acceptable salts thereof as active ingredient:

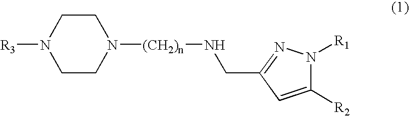

wherein:

R₁ is hydrogen, C₁-C₆ alkyl or phenyl;

R₂ is hydrogen, C₁-C₆ alkyl, phenyl, or furan;

R₃ is phenyl or benzhydryl;

wherein the phenyl or benzhydryl may be optionally substituted by a substituent selected from halogen, C₁-C₆ alkyl and piperidine; and

n is an integer from 1 to 6.

More preferably,

R₁ is hydrogen; methyl, ethyl, propyl, isopropyl, butyl, isobutyl, cyclobutyl, pentyl, isopentyl, cyclopentyl, hexyl, isohexyl, cyclohexyl; phenyl; or phenyl substituted by a substituent selected from methyl, ethyl, propyl, isopropyl, butyl, isobutyl, cyclobutyl, pentyl, isopentyl, cyclopentyl, hexyl, isohexyl and cyclohexyl,

R₂ is hydrogen; methyl, ethyl, propyl, isopropyl, butyl, isobutyl, cyclobutyl, pentyl, isopentyl, cyclopentyl, hexyl, isohexyl, cyclohexyl; phenyl; phenyl substituted by a substituent selected from methyl, ethyl, propyl, isopropyl, butyl, isobutyl, cyclobutyl, pentyl, isopentyl, cyclopentyl, hexyl, isohexyl, cyclohexyl and piperidine; or furan,

R₃ is phenyl; phenyl substituted by halogen; benzhydryl or benzhydryl substituted by halogen, and

n is an integer from 1 to 6.

Preferred specific examples of the pyrazole derivative represented by Formula 1 include:
1-phenyl-3-{2-[4-(3-chlorophenyl)piperazin-1-yl]ethyl}aminomethyl-5-(2-furyl)pyrazole,
3-{2-[4-(3-chlorophenyl)piperazin-1-yl]ethyl}aminomethyl-5-methylpyrazole,
1-phenyl-3-{2-[4-(4-chlorophenyl)piperazin-1-yl]ethyl}aminomethyl-5-methylpyrazole,
1-t-butyl-3-{2-[4-(4-chlorophenyl)piperazin-1-yl]ethyl}aminomethyl-5-propylpyrazole,
1-phenyl-3-{2-[4-(4-chlorophenyl)piperazin-1-yl]ethyl}aminomethyl-5-propylpyrazole,
1-phenyl-3-{2-[4-(4-chlorophenyl)piperazin-1-yl]ethyl}aminomethyl-5-isobutylpyrazole,
1-t-butyl-3-{2-[4-(4-chlorophenyl)piperazin-1-yl]ethyl}aminomethyl-5-isobutylpyrazole,
1-phenyl-3-{2-[4-(4-chlorophenyl)piperazin-1-yl]ethyl}aminomethyl-5-(2-furyl)pyra zole,
1-t-butyl-3-{2-[4-(4-chlorophenyl)piperazin-1-yl]ethyl}aminomethyl-5-toluoylpyrazole,
1-phenyl-3-{2-[4-(4-chlorophenyl)piperazin-1-yl]ethyl}aminomethyl-5-4-(N-piperidyl)phenylpyrazole,
1,5-diphenyl-3-{2-[4-(4-chlorophenyl)piperazin-1-yl]ethyl}aminomethyl-5-pyrazole,
1,5-diphenyl-3-{2-[4-(4-chlorophenyl)piperazin-1-yl]ethyl}aminomethyl-5-toluoylpyrazole, and
1-phenyl-3-{2-[4-(4-chlorobenzhydryl)piperazin-1-yl]ethyl}aminomethyl-5-toluoylpyrazole.

The pyrazole derivatives represented by Formula 1 are compounds known in the art and can be prepared by a variety of well-known preparation methods [Korean Patent No. 0654328]. Thus, the present invention is characterized not by the derivatives or the preparation methods thereof, but by their superior inhibition effect against the 5-HT₃A receptor.

The present invention can be applied to both the prevention and treatment of CNS diseases through antagonistic action against the 5-HT₃A receptor, specifically, emesis, nausea, alcoholism, drug abuse, depression, compulsive neurosis, anxiety, seizure, Alzheimer's disease, Parkinson's disease, Huntington's chorea, psychosis, schizophrenia, suicidal tendency, sleep disorder, appetite disorder, migraine, and the like.

The pyrazole derivative represented by Formula 1 may be used in the form of a pharmaceutically acceptable salt. Preferably, the salt may be an acid adduct salt formed from a pharmaceutically acceptable free acid. The pyrazole derivative represented by Formula 1 may be prepared in to a pharmaceutically acceptable acid adduct salt by the method commonly used in the related art. The free acid may be an organic or inorganic acid. Examples of the inorganic acid include hydrochloric acid, bromic acid, sulfuric acid, phosphoric acid, and the like. Examples of the organic acid include citric acid, acetic acid, lactic acid, tartaric acid, maleic acid, fumaric acid, formic acid, propionic acid, oxalic acid, trifluoroacetic acid, benzoic acid, gluconic acid, methanesulfonic acid, glycolic acid, succinic acid, 4-toluenesulfonic acid, galacturonic acid, embonic acid, glutamic acid, aspartic acid, and the like.

When the composition of the present invention is used as medicine, the pyrazole derivative represented by Formula 1 or a pharmaceutically acceptable salt thereof may be mixed with a vehicle commonly used in the pharmaceutical field and prepared into common preparation forms, for example, oral administration formulations such as tablet, capsule, troche, liquid, suspension, and the like; injection formulations such as solution or suspension for injection, dry powder for injection which can be used immediately by dissolving in distilled water for injection, and the like; or various other formulations including ointment. The pharmaceutical formulations prepared by using a commonly used vehicle may be administered orally or parenterally, for example, intravenously, subcutaneously, intra-abdominally or locally. The dose of the pyrazole derivative represented by Formula 1 according to the present invention may vary depending on the patient's age, physical conditions, and the like. In general, For an adult, 10 to 500 mg, preferably 50 to 300 mg, is administered per day. Depending on the decision of a pharmacist or a doctor, it may be administered several times, preferably once to 6 times, a day.

As used herein, health food refers to a food prepared into capsule, powder, suspension, and the like by adding the pyrazole derivative represented by Formula 1. It is intended to provide specific good effects on health, but, differently from medicines, it is made from food and is without adverse reactions, which may occur during long-term use of medicines.

Specifically, the food may be confectionery, processed foods, dairy products, drinks, or the like, and may be in any form, without particular limitation. For example, the health food may be in the form of solid, semisolid, gel, liquid, powder, and the like.

For clinical purposes, the composition containing the pyrazole derivative represented by Formula 1 as active ingredient may be administered orally or parenterally. The composition may be in the form of general medicines, quasi-drugs, health foods, and the like.

### EXAMPLES

The following examples are provided to further illustrate the present invention, and are not intended to limit the same.

The pyrazole derivatives of the following examples are disclosed in the literature Korean Patent No. 0654328; and U.S. patent application Ser. No. 11/509,769, and can be prepared by methods known in the art. Activity screening was carried out in order to confirm the use of the compounds for treatment of diseases as described herein.

### Preparation Examples

### 1-Phenyl-3-{2-[4-(3-chlorophenyl)piperazin-1-yl]ethyl}aminomethyl-5-(2-furyl)pyrazole (Compound 1)

50 mg (0.208 mmol) of 2-[4-(3-chlorophenylpiperazin)-1-yl]-ethylamine and 25.24 mg (0.104 mmol) of 1-phenyl-(2-furyl)pyrazole-5-aldehyde were dissolved in 5 mL of purified CH₂Cl₂. After putting in a 4 Å molecular sieve (5 beads), stirring was carried out at room temperature for 12 hours. Then, after adding 66.28 mg (0.313 mmol) of NaBH(OAc)₃, stirring was carried out at room temperature for 1 hour. Progress of reaction was confirmed by TLC(CH₂Cl₂:MeOH=5:1). After the reaction was completed, water was added to the reaction mixture and the water layer was extracted with CH₂Cl₂. The organic layer was dried with anhydrous MgSO₄ and concentrated under reduced pressure after filtration. The concentrate was separated by column chromatography (CH₂Cl₂:MeOH=10:1). Compound 1 was obtained.

Yield: 68.6%

¹H NMR (300 MHz, CDCl₃) δ 7.32-7.56 (m, 6H), 7.13 (t, J=7.8 Hz, 1H), 6.71-6.90 (m, 3H), 6.67 (s, 1H), 6.33 (m, 1H), 5.97 (m, 1H), 3.99 (s, 2H), 3.13 (m, 4H), 2.90 (m, 2H), 2.48-2.71 (m, 6H)

The following compounds were synthesized in the same manner as described above, from piperazylamine and pyrazolealdehyde.

### 3-{2-[4-(3-Chlorophenyl)piperazin-1-yl]ethyl}aminomethyl-5-methylpyrazole (Compound 2)

Yield: 60.4%

¹H NMR (300 MHz, CDCl₃) δ 7.16 (t, J=8.1 Hz, 1H), 6.73-6.87 (m, 3H), 6.17 (s, 1H), 4.90 (brs, 1H), 3.99 (s, 2H), 3.17 (m, 4H), 3.00 (t, J=5.8 Hz, 2H), 2.73 (m, 4H), 2.60 (m, 2H), 2.04 (2, 3H)

### 1-Phenyl-3-{2-[4-(4-chlorophenyl)piperazin-1-yl]ethyl}aminomethyl-5-methylpyrazole (Compound 3)

Yield: 71%

¹H NMR (300 MHz, CDCl₃) δ 7.33-7.46 (m, 8H), 7.12-7.32 (m, 7H), 6.27 (s, 1H), 4.22 (s, 1H), 3.98 (s, 2H), 2.93 (t, J=6.0 Hz, 2H), 2.64 (t, J=6.0 Hz, 2H), 2.50 (m, 4H), 2.38 (m, 4H), 2.29 (s, 3H)

¹³C NMR (75 MHz, CDCl₃) δ 148.6, 142.7, 140.1, 139.5, 129.0, 128.4, 127.8, 127.7, 126.9, 124.8, 106.4, 76.1, 56.0, 53.2, 51.6, 45.9, 44.6, 12.3

IR (KBr, cm⁻¹) 3356 (—NH), 2924, 2810, 1502, 1452, 1008

FABHRMS m/z calcd for C₃₀H₃₆N₅ (M+H)⁺ 466.2971, Found 466.2983

### 1-t-Butyl-3-{2-[4-(4-chlorophenyl)piperazin-1-yl]ethyl}aminomethyl-5-propylpyrazole (Compound 4)

Yield: 70%

¹H NMR (300 MHz, CDCl₃) δ 7.42 (d, J=7.1 Hz, 4H), 7.28 (t, J=7.3 Hz, 4H), 7.18 (t, J=7.3 Hz, 2H), 6.01 (s, 1H), 4.23 (s, 1H), 3.91 (s, 2H), 2.76 (t, J=6.0 Hz, 2H), 2.36-2.59 (m, 12H), 1.52-1.68 (m, 11H), 0.95 (t, J=7.4 Hz, 3H)

¹³C NMR (75 MHz, CDCl₃) δ 149.9, 142.7, 141.6, 128.4, 127.9, 126.9, 105.5, 59.5, 57.7, 53.5, 51.9, 46.7, 46.0, 30.3, 23.0, 14.0

mp=81 to 81° C.

### 1-Phenyl-3-{2-[4-(4-chlorophenyl)piperazin-1-yl]ethyl}aminomethyl-5-propylpyrazole (Compound 5)

Yield: 52%

¹H NMR (300 MHz, CDCl₃) δ 7.33-7.39 (m, 6H), 7.21-7.32 (m, 3H), 6.21 (s, 1H), 3.89 (s, 2H), 2.89 (m, 2H), 2.23-2.75 (m, 12H), 1.45 (m, 2H), 0.90 (t, J=7.0 Hz, 3H)

### 1-Phenyl-3-{2-[4-(4-chlorophenyl)piperazin-1-yl]ethyl}aminomethyl-5-isobutylpyrazole (Compound 6)

Yield: 53%

¹H NMR (300 MHz, CDCl₃) δ 7.36-7.50 (m, 6H), 7.15-7.34 (m, 3H), 6.19 (s, 1H), 3.89 (s, 2H), 3.15 (m, 2H), 2.68 (m, 2H), 2.31-2.61 (m, 10H), 1.78 (m, 1H), 0.86 (d, J=6.9 Hz, 6H)

### 1-t-Butyl-3-{2-[4-(4-chlorophenyl)piperazin-1-yl]ethyl}aminomethyl-5-isobutylpyrazole (Compound 7)

Yield: 28%

¹H NMR (300 MHz, CDCl₃) δ 7.25-7.37 (m, 4H), 6.09 (s, 1H), 3.51 (s, 2H), 3.18 (m, 4H), 2.74 (t, J=5.6 Hz, 2H), 2.62 (t, J=5.6 Hz, 2H), 2.45-2.59 (m, 6H), 1.80 (m, 1H), 1.44 (s, 9H), 0.90 (d, J=7.2 Hz, 6H)

mp=133-134° C.

### 1-Phenyl-3-{2-[4-(4-chlorophenyl)piperazin-1-yl]ethyl}aminomethyl-5-(2-furyl)pyrazole (Compound 8)

Yield: 48%

¹H NMR (300 MHz, CDCl₃) δ 7.32-7.49 (m, 6H), 7.18-7.21 (m, 4H), 6.65 (s, 1H), 6.31 (m, 1H), 5.95 (m, 1H), 3.94 (s, 2H), 3.15 (m, 2H), 2.91 (t, J=6.0 Hz, 4H), 2.69 (t, J=6.0 Hz, 4H), 2.50 (m, 2H)

### 1-t-Butyl-3-{2-[4-(4-chlorophenyl)piperazin-1-yl]ethyl}aminomethyl-5-toluoylpyrazole (Compound 9)

Yield: 81%

¹H NMR (300 MHz, CDCl₃) δ 7.20-7.31 (m, 5H), 7.07-7.19 (m, 8H), 5.74 (s, 1H), 3.78 (s, 2H), 3.06 (m, 2H), 2.77 (m, 4H), 2.50-2.68 (m, 6H), 2.38 (s, 3H)

### 1-Phenyl-3-{2-[4-(4-chlorophenyl)piperazin-1-yl]ethyl}aminomethyl-5-4-(N-piperidyl)phenylpyrazole (Compound 10)

Yield: 56%

¹H NMR (300 MHz, CDCl₃) δ 7.19-7.35 (m, 8H), 6.96-7.14 (m, 6H), 6.00 (s, 1H), 3.98 (s, 2H), 3.16 (m, 4H), 3.00 (t, J=5.9 Hz, 2H), 2.70 (m, 6H), 2.48 (m, 4H), 1.56-1.76 (m, 6H)

### 1,5-Diphenyl-3-{2-[4-(4-chlorophenyl)piperazin-1-yl]ethyl}aminomethyl-5-pyrazole (Compound 11)

Yield: 75%

¹H NMR (300 MHz, CDCl₃) δ 7.11-7.36 (m, 14H), 6.03 (s, 1H), 3.97 (s, 2H), 3.16 (m, 4H), 2.94 (t, J=6.0 Hz, 2H), 2.68 (m, 4H), 2.50 (m, 2H)

### 1,5-Diphenyl-3-{2-[4-(4-chlorophenyl)piperazin-1-yl]ethyl}aminomethyl-5-toluylpyrazole (Compound 12)

Yield: 81%

¹H NMR (300 MHz, CDCl₃) δ 7.20-7.31 (m, 5H), 7.07-7.19 (m, 8H), 5.74 (s, 1H), 3.78 (s, 2H), 3.06 (m, 2H), 2.77 (m, 4H), 2.50-2.68 (m, 6H), 2.38 (s, 3H)

### 1-Phenyl-3-{2-[4-(4-chlorobenzhydryl)piperazin-1-yl]ethyl}aminomethyl-5-toluylpyrazole (Compound 13)

Yield: 41%

¹H NMR (300 MHz, CDCl₃) δ 7.32-7.40 (m, 4H), 7.18-7.31 (m, 5H), 7.13 (d, J=5.8 Hz, 2H), 7.08 (d, J=6.3 Hz, 2H), 6.15 (s, 1H), 4.18 (s, 1H), 4.06 (s, 2H), 2.72 (t, J=5.6 Hz, 4H), 2.49 (m, 4H), 2.32-2.45 (m, 7H), 1.38 (s, 9H)

¹³C NMR (75 MHz, CDCl₃) δ 141.9, 141.1, 138.5, 132.6, 130.4, 130.1129.1, 128.7, 128.6, 128.5, 128.4, 127.7, 127.2, 108.8, 61.5, 52.8, 51.6, 45.0, 43.5, 31.1, 31.1, 29.7, 21.3

IR (KBr, cm⁻¹) 3356 (—NH), 2924, 2814, 1450, 1010, 912, 806

FABHRMS m/z calcd for C₃₄H₄₄ClN₅ (M+H)⁺ 556.3207, Found 556.3207

### Example 1

### Assay for 5-HT₃A receptor

Antagonistic activity of the pyrazole derivatives represented by Formula 1 against the 5-HT₃A receptor was identified.

Although the results for exemplary pyrazole derivatives represented by Formula 1 listed in Table 1 are given, other compounds represented by Formula 1 are expected to exhibit identical or similar antagonistic activity against the 5-HT₃A receptor.

1) Culturing and Isolation of Xenopus Oocytes Xenopus laevis (African clawed frog) was anesthetized with ice and follicular oocytes were taken out after incising the lateral abdominal region. The follicular oocytes were washed with Ca²⁺-free OR2 solution (82.5 mM NaCl, 2 mM KCl, 1 mM MgCl₂, 5 mM HEPES buffer, 2.5 mM sodium pyruvate, penicillin 100 units/mL and streptomycin 100 μg/mL). Then, after addition of 2 mg/mL collagenase to the solution followed by about 2 hours of gentle shaking, only the oocytes of stages V and VI which have lost their follicles were selected. These oocytes were washed several times with ND96 media [pH 7.5 solution containing 96 mM NaCl, 2 mM KCl, 1 mM MgCl₂, 1.8 mM CaCl₂, 5 mM HEPES, 0.5 mM theophylline and 50 μg/mL gentamycin] and cultured in an incubator of 16 to 18° C. The medium was exchanged every day, and all the experiments were performed within 2 to 6 days after the isolation of the oocytes.

2) Recording of 5-HT₃A Receptor Channel Activity

The oocytes were placed in a small 0.5 mL Plexiglas net chamber and superfused in a solution (ND96 medium) containing the test compound or not. Microelectrodes were filled with 3 M KCl and resistance was adjusted to about 0.2 to 0.7 MΩ. Most of the electrophysiological experiments were carried out while holding the oocytes such that the holding potential was maintained at −100 mV for a duration of 2000 seconds. During the current-voltage experiments, the voltage was increased from −100 mV to +60 mV, by 10 mV. During the time course experiments, Na⁺ current was induced by evoking from −100 mV to −10 mV for 20 ms. Two-electrode voltage-clamp recordings were carried out using an oocyte clamp (OC-725C, Warner Instrument) connected to Digidata 1200B [Choi, S., Jung, S. Y., Lee, J. H., Sala, F., Criado, M., Mulet, J., Valor, L. M., Sala, S., Engel, A. G. and Nah, S. Y. (2002) Eur. J. Pharmacol. 442, 37-45].

3) Oocyte Injection

cRNAs of 5-HT₃A receptors existing in the nervous system (3 to 5 ng/oocyte, each) were injected into the oocytes. Oocyte injection was carried out using Nanoject Automatic Oocyte Injector (Drummond Scientific, Broomall, Pa.), 40 to 50 mL at once. Oocyte volume was assumed to be approximately 1 μL.

4) Expression of 5-HT₃A Receptor in Xenopus Oocytes

E. coli was transformed with the plasmids having the 5-HT₃A receptor channels existing in the nervous system. Then, cDNAs having 5-HT₃A receptors were prepared using Miniprep kit (Promega). After linearization using restriction enzymes, cRNAs for 5-HT₃A receptor channels were prepared using in vitro transmission kit (Promega or Ambion).

50% inhibitory concentration (IC₅₀), maximum inhibition value (Vₘₐₓ) and Hill coefficient (nH) of the pyrazole derivatives prepared above for the serotonin 5-HT₃A receptor were calculated. The result is given in the following Table 1. Concentrations of the compounds were 0.01, 0.03, 0.1, 0.3, 1, 3, 10 and 100 μM, or 0.003, 0.01, 0.03, 0.1, 0.3, 1, 3 and 10 μM, depending on their efficiency. IC₅₀, Vₘₐₓ and Hill coefficient for each compound were obtained from concentration-response curves.

TABLE 1

| | Compounds | Vₘₐₓ | IC₅₀ (μM) | nH |
|---|---|---|---|---|
| 1 | 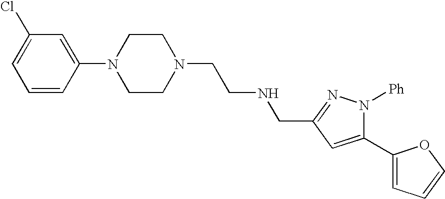 | 95.9 ± 2.8 | 9.4 ± 0.7 | 1.5 ± 0.2 |
| 2 | 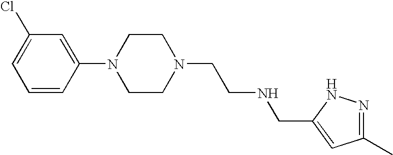 | 90.6 ± 2.7 | 9.6 ± 1.1 | 1.0 ± 0.1 |
| 3 | 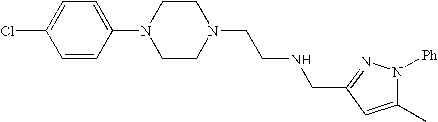 | 98.5 ± 1.2 | 2.5 ± 0.3 | 1.4 ± 0.1 |
| 4 | 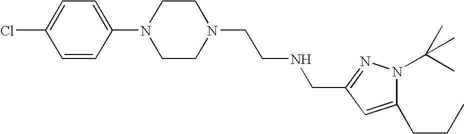 | 97.3 ± 5.6 | 1.3 ± 0.3 | 1.4 ± 0.3 |
| 5 | 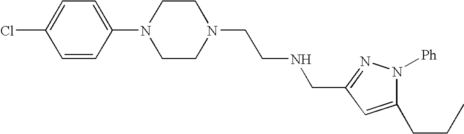 | 94.8 ± 4.8 | 1.3 ± 0.2 | 1.5 ± 0.3 |
| 6 | 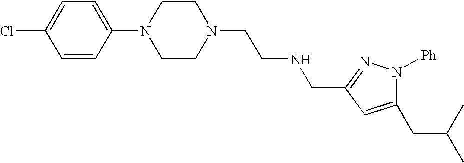 | 102.8 ± 2.5 | 2.8 ± 0.2 | 1.2 ± 0.1 |
| 7 | 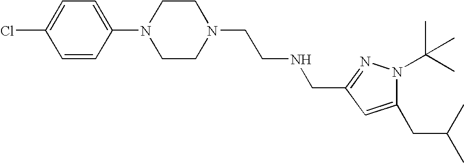 | 104.3 ± 3.5 | 3.3 ± 0.9 | 1.4 ± 0.4 |
| 8 | 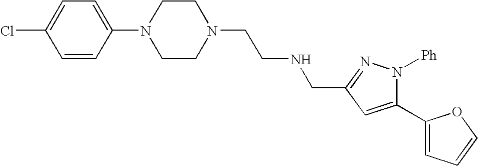 | 97.3 ± 2.0 | 4.3 ± 0.3 | 2.0 ± 0.2 |
| 9 | 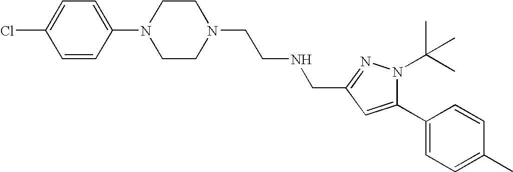 | 102.6 ± 6.5 | 2.4 ± 0.4 | 1.4 ± 0.3 |
| 10 | 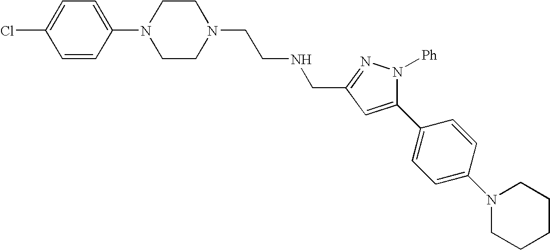 | 95.9 ± 1.6 | 6.4 ± 0.3 | 1.9 ± 0.1 |
| 11 | 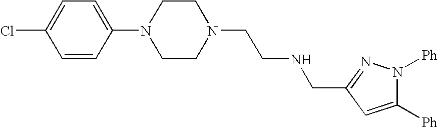 | 101.3 ± 2.6 | 3.1 ± 0.3 | 1.1 ± 0.1 |
| 12 | 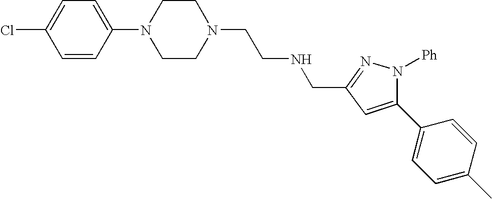 | 95.5 ± 3.5 | 2.2 ± 0.2 | 1.8 ± 0.3 |
| 13 | 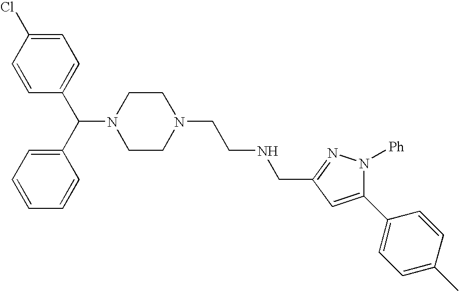 | 73.1 ± 3.1 | 8.2 ± 0.8 | 2.2 ± 0.4 |
| Control | MDL72222 | 99.6 ± 7.7 | 0.77 ± 0.16 | 1.25 ± 0.27 |

As seen in Table 1, the Xenopus laevis oocytes treated with the compounds of the present invention were confirmed to inhibit the inward current (I5-HT) induced by 5-HT (1 μM), when measured by the two-electrode voltage clamp assay technique.

Specifically, the compounds of the present invention were confirmed to have superior activity against CNS diseases related with the serotonin 5-HT₃A receptor, as seen by the measurement result of Vₘₐₓ, IC₅₀ and nH. The result is almost comparable to MDL72222, which is currently used in the pharmaceutical field. Therefore, the compounds of the present invention are useful in treating CNS diseases related with the serotonin 5-HT₃A receptor.

### Example 2

### Toxicity Test

In order to identify toxicity of the pyrazole derivatives represented by Formula 1 according to the present invention, 1 to 20 mg of the compounds were intra-abdominally administered to 24 mice. Behaviors of the mice were monitored for 24 hours and survival was observed 24 hours later.

As a result, the pyrazole derivatives of the present examples had no toxicity problem at all. Of the six mice to which 20 mg was administered, three survived and the other three were sacrificed. In contrast, all the mice survived when the administration dose was less than 20 mg. No statistically significant change was observed in behavior monitoring between the mice to which the compounds were administered and not. Based on the experiment result, the toxic dose of the pyrazole derivatives at which approximately half of the mice survive (TD₅₀) can be calculated at 20 mg (1 mg/g).

### Example 3

### Preparation of Tablet

Tablets for oral administration were prepared by wet granulation and dry granulation using the pyrazole derivative represented by Formula 1 according to the present invention as active ingredient.

Composition:

Active ingredient 200 mg, light anhydrous silica acid 10 mg, magnesium stearate 2 mg, microcrystalline cellulose 50 mg, sodium starch glycolate 25 mg, cornstarch 113 mg, anhydrous ethanol adequate.

### Example 4

### Preparation of Ointment

Ointments were prepared using the pyrazole derivative represented by Formula 1 according to the present invention as active ingredient.

Composition:

Active ingredient 5 g, cetyl palmitate 20 g, cetanol 40 g, isopropyl myristate 80 g, sorbitan monostearate 20 g, polysorbate 60 g, propyl p-hydroxybenzoate 1 g, methyl p-hydroxybenzoate 1 g, phosphoric acid and purified water adequate.

### Example 5

### Preparation of Injection

Injections were prepared using the pyrazole derivative represented by Formula 1 according to the present invention as active ingredient.

Composition:

Active ingredient 100 mg, mannitol 180 mg, sodium hydrogen phosphate 25 mg, injection for water 2974 mg

### Example 6

### Preparation of Drink

Drink compositions were prepared by dissolving 500 mg of the pyrazole derivative represented by Formula 1 according to the present invention in adequate amount of water, vitamin C, as supplementary ingredient, adding adequate amount of citric acid, sodium citrate and oligosaccharide, as favor enhancer, and adding adequate amount of sodium benzoate, as preservative, and further adding water to make 100 mL. As occasion demands, taurine, myoinositol, folic acid, pantothenic acid, etc. may be added alone or in combination.

As described above, the pyrazole derivatives represented by Formula 1 according to the present invention have superior 5-HT₃A inhibition and binding activities and can be used for the treatment of CNS diseases as antagonist against the 5-HT₃A receptor.

Accordingly, the pyrazole derivatives represented by Formula 1 according to the present invention are effective as pharmaceutical compositions, medicines and health foods for the prevention and treatment of CNS diseases, including emesis, nausea, alcoholism, drug abuse, depression, compulsive neurosis, anxiety, seizure, Alzheimer's disease, Parkinson's disease, Huntington's chorea, psychosis, schizophrenia, suicidal tendency, sleep disorder, appetite disorder and migraine.

Although the above embodiments of the present invention have been disclosed for illustrative purposes, those skilled in the art will appreciate that various modifications, additions and substitutions may be made, without departing from the scope and spirit of the present invention.

What is claimed:

1. A pharmaceutical composition for preventing and/or treating CNS diseases related to serotonin 5-HT₃A receptor, which comprises a) one or more pyrazole derivatives represented by formula (I) or a pharmaceutically-acceptable salt or salts thereof as an active ingredient: 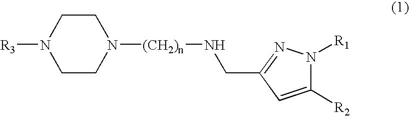
wherein
R₁ is hydrogen, C₁-C₆ alkyl or phenyl;
R₂ is hydrogen, C₁-C₆ alkyl, phenyl or furan;
R₃ is phenyl or benzhydryl;
wherein said phenyl or benzhydryl is each optionally substituted by a substituent selected from the group consisting of halogen, C₁-C₆ alkyl and piperidine; and
n is an integer from 1 to 6; and
b) a pharmaceutically-acceptable carrier.

2. The composition of claim 1, wherein
R₁ is hydrogen; methyl, ethyl, propyl, isopropyl, butyl, isobutyl, cyclobutyl, pentyl, isopentyl, cyclopentyl, hexyl, isohexyl, cyclohexyl; phenyl; or phenyl substituted by a substituent selected from methyl, ethyl, propyl, isopropyl, butyl, isobutyl, cyclobutyl, pentyl, isopentyl, cyclopentyl, hexyl, isohexyl and cyclohexyl,
R₂ is hydrogen; methyl, ethyl, propyl, isopropyl, butyl, isobutyl, cyclobutyl, pentyl, isopentyl, cyclopentyl, hexyl, isohexyl, cyclohexyl; phenyl; phenyl substituted by a substituent selected from methyl, ethyl, propyl, isopropyl, butyl, isobutyl, cyclobutyl, pentyl, isopentyl, cyclopentyl, hexyl, isohexyl, cyclohexyl and piperidine; or furan,
R₃ is phenyl; phenyl substituted by halogen; benzhydryl or benzhydryl substituted by halogen, and
n is an integer from 1 to 6.

3. The composition of claim 1, wherein the pyrazole derivative is selected from the group consisting of
1-phenyl-3-{2-[4-(3-chlorophenyl)piperazin-1-yl]ethyl}aminomethyl-5-(2-furyl)pyrazole,
3-{2-[4-(3-chlorophenyl)piperazin-1-yl]ethyl}aminomethyl-5-methylpyrazole,
1-phenyl-3-{2-[4-(4-chlorophenyl)piperazin-1-yl]ethyl}aminomethyl-5-methylpyrazole,
1-t-butyl-3-{2-[4-(4-chlorophenyl)piperazin-1-yl]ethyl}aminomethyl-5-propylpyrazole,
1-phenyl-3-{2-[4-(4-chlorophenyl)piperazin-1-yl]ethyl}aminomethyl-5-propylpyrazole,
1-phenyl-3-{2-[4-(4-chlorophenyl)piperazin-1-yl]ethyl}aminomethyl-5-isobutyl pyrazole,
1-t-butyl-3-{2-[4-(4-chlorophenyl)piperazin-1-yl]ethyl}aminomethyl-5-isobutylpyrazole,
1-phenyl-3-{2-[4-(4-chlorophenyl)piperazin-1-yl]ethyl}aminomethyl-5-(2-furyl)pyra zole,
1-t-butyl-3-{2-[4-(4-chlorophenyl)piperazin-1-yl]ethyl}aminomethyl-5-toluoylpyrazole,
1-phenyl-3-{2-[4-(4-chlorophenyl)piperazin-1-yl]ethyl}aminomethyl-5-4-(N-piperidyl)phenylpyrazole,
1,5-diphenyl-3-{2-[4-(4-chlorophenyl)piperazin-1-yl]ethyl}aminomethyl-5-pyrazole,
1,5-diphenyl-3-{2-[4-(4-chlorophenyl)piperazin-1-yl]ethyl}aminomethyl-5-toluoylpyrazole, and
1-phenyl-3-{2-[4-(4-chlorobenzhydryl)piperazin-1-yl]ethyl}aminomethyl-5-toluoylpyrazole.

4. The composition of claim 1, wherein the CNS diseases are selected from the group consisting of emesis, nausea, alcoholism, drug abuse, depression, compulsive neurosis, anxiety, seizure, Alzheimer's disease, Parkinson's disease, Huntington's chorea, psychosis, schizophrenia, suicidal tendency, sleep disorder, appetite disorder and migraine.

5. The composition of claim 1, which is in a form of a tablet.

6. The composition of claim 1, which is in a form of an ointment.

7. The composition of claim 1, which is in a form suitable for injection.

8. A health food comprising a pyrazole derivative represented by formula (I) or a pharmaceutically acceptable salt or salts thereof as active ingredient: 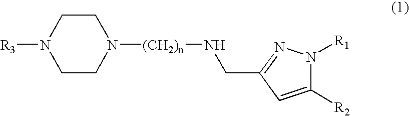
wherein R₁, R₂ and R₃ are as defined in claim 1.

9. The health food of claim 8. which is in a form of a beverage.

10. A method of treating a CNS disease related to 5-HT₃A receptor in a mammal, which comprises administering to the mammal, an amount of the composition of claim 1, effective to treat the CNS disease.

11. The method of claim 10, wherein said CNS disease is selected from the group consisting of emesis, nausea, alcoholism, drug abuse, depression, compulsive neurosis, anxiety, seizure, Alzheimer's disease, Parkinson's disease, Huntington's chorea, psychosis, schizophrenia, suicidal tendency's, sleep disorder, appetite disorder and migraine.

12. A method of effecting antagonistic activity against serotonin 5-H₃TA in a mammal, which comprises administering to the mammal, an amount of the composition of claim 1, effective to cause the antagonistic activity.

13. A method of treating a CNS disease related to 5-HT₃A receptor in mammal, which comprises administering to the mammal, an amount of one or more pyrazole derivatives of the formula (I) or a pharmaceutically-acceptable salt or salts thereof: 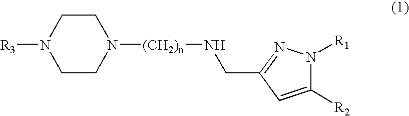
wherein
R₁ is hydrogen, C₁-C₆ alkyl or phenyl;
R₂ is hydrogen, C₁-C₆ alkyl, phenyl or furan;
R₃ is a phenyl or benzhydryl;
wherein said phenyl or benzhydryl is each optically substituted by a substituent selected from the group consisting of halogen, C₁-C₆ alkyl and piperidine; and;
n is an integer from 1 to 6.

14. The method of claim 13, wherein in the compound of the formula (I),
R₁ is hydrogen; methyl, ethyl, propyl, isopropyl, butyl, isobutyl, cyclobutyl, pentyl, isopentyl, cyclopentyl, hexyl, isohexyl, cyclohexyl; phenyl; or phenyl substituted by a substituent selected from methyl, ethyl, propyl, isopropyl, butyl, isobutyl, cyclobutyl, pentyl, isopentyl, cyclopentyl, hexyl, isohexyl and cyclohexyl,
R₂ is hydrogen; methyl, ethyl, propyl, isopropyl, butyl, isobutyl, cyclobutyl, pentyl, isopentyl, cyclopentyl, hexyl, isohexyl, cyclohexyl; phenyl; phenyl substituted by a substituent selected from methyl, ethyl, propyl, isopropyl, butyl, isobutyl, cyclobutyl, pentyl, isopentyl, cyclopentyl, hexyl, isohexyl, cyclohexyl and piperidine; or furan,
R₃ is phenyl; phenyl substituted by halogen; benzhydryl or benzhydryl substituted by halogen, and
n is an integer from 1 to 6.

15. The method of claim 10, wherein the mammal is a human.

16. The method of claim 12, wherein the mammal is a human.

17. The method of claim 13, wherein the mammal is a human.

18. A method of effecting antagonistic activity against one or more pyrazole derivatives of the formula (I) or a pharmaceutically-acceptable salt or salts thereof; 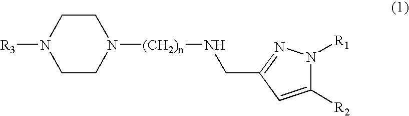
wherein
R₁ is hydrogen, C₁-C₆ alkyl or phenyl;
R₂ is hydrogen, C₁-C₆ alkyl, phenyl or furan;
R₃ is a phenyl or benzhydryl;
wherein said phenyl or benzhydryl is each optically substituted by a substituent selected from the group consisting of halogen, C₁-C₆ alkyl and piperidine; and;
n is an integer from 1 to 6.
